(19) United States
(12) Patent Application Publication
(10) Pub. No.: US 2007/0110742 A1
(43) Pub. Date: May 17, 2007

(54) Recombinant peptide vector comprising the gene for treatment for autoimmune diseases

(76) Inventors: Young-Jin Chae, Gyeonggi-do (KR); Eun-Hwa Choi, Seoul (KR)

Correspondence Address: BUCHANAN, INGERSOLL & ROONEY PC, POST OFFICE BOX 1404, ALEXANDRIA, VA, 22313-1404, US

(21) Appl. No.: 10/581,041

(22) PCT Filed: Nov. 29, 2003
(86) PCT No.: PCT/KR03/02613

§ 371 Date: May 30, 2006

(51) Int. Cl. A61K 39/395; C07H 21/04; C12P 21/06; C12N 5/06

(52) U.S. Cl. 424/131.1; 435/69.1; 435/320.1; 435/327; 530/387.2; 530/391.1; 424/178.1; 536/23.53

(57) ABSTRACT

The present invention provides a recombinant peptide vector comprising a leader peptide, linker DNAs and a DNA construct formed by operably linking expression control sequences with a therapeutic gene encoding a fusion protein where the extracellular domain of CTLA4 is bound to the Fc fragment of immunoglobulin, wherein the leader peptide is linked to both ends of the DNA construct by the linker DNAs. Also, the present invention provides a method for preparing the recombinant peptide vector, and a pharmaceutical composition for the treatment of autoimmune diseases, which comprises a pharmaceutically effective amount of the recombinant peptide vector, and a pharmaceutically acceptable carrier.

### TECHNICAL FIELD

The present invention relates to a recombinant peptide vector containing a gene for the treatment of autoimmune diseases. More particularly, the present invention relates to a recombinant peptide vector (FIG. 1) comprising a leader peptide, linker DNAs and a DNA construct formed by operably linking expression control sequences with a therapeutic gene encoding a fusion protein where the extracellular domain of CTLA4 is bound to the Fc fragment of immunoglobulin, wherein the leader peptide is linked to both ends of the DNA construct by the linker DNAs. In addition, the present invention relates to a preparation method thereof and a composition for the treatment of autoimmune diseases, particularly systemic lupus erythematosus, which comprises a pharmaceutically effective amount of the recombinant vector, and a pharmaceutically acceptable carrier.

### BACKGROUND ART

Systemic lupus erythematosus (or lupus) is an autoimmune disease that causes symptoms, such as nonerosive polyarthritis, glomerulonephropathy, hemolytic anemia, thrombocytopenia, neutropenia, polymyositis, persistent/recurring fever, and facia/mucocutaneous dermatitis.

Lupus has a difference in symptoms between patients and shows various symptoms ranging from mild to severe symptoms, and thus, it should be suitably treated for its symptoms. For skin symptoms or arthritis, an antimalarial formulation is generally used, and when anti-inflammatory action is required, a small amount of adrenocortical hormone is sometimes used. For the treatment of kidney diseases or severe symptoms, such as severe thrombocytopenia or stroke, a high dose of adrenocortical hormones or immune suppressors are used. Recently, in addition to such prior general therapies, gene therapies using a native or artificially constructed gene capable of suppressing an immune response are also attempted.

For such gene therapies, viral vectors have been mainly used to transfer a gene with therapeutic effect into cells. However, although various viral vectors have been developed and the clinical tests thereof have been made until now, the use of such vectors is limited due to the characteristics of virus. Virus binds to a certain ligand in a cell membrane, thereby entering cells. Accordingly, the cells which can be infected are limited depending on the kind of ligands to which virus can be bound. Thus, diseases against which the viral vector can be applied will inevitably be limited.

Another important reason for the limited usage of the viral vectors is that all viruses cause immune responses to a host (in acute conditions, inflammatory response, and in chronic conditions, the production of an antibody to vectors) when they enter a living body, although there is a difference in the response degree. Accordingly, the great loss of viruses by the immune response is caused and antibodies to the vectors are produced, thereby the vectors have limitations in secondary inoculation (their readministration has no effect). In addition, there are problems, such as toxicity, the size of a therapeutic gene, in vivo duration, and integration into chromosome of host cell, depending on the kind of virus. For these reasons, although 20 years have passed since virus factors turned out to be applicable in gene therapy, the viral vectors are clinically used in very limited applications.

Accordingly, the present inventors previously developed a non-viral peptide vector overcoming all the above-described problems (Patent Application Nos: Korea 10-2001-6587; U.S. Ser. No. 10/071,476; Japan 2002-032708; China 02104729.4; and Europe 02002623) and used it for the transfer of a specific therapeutic gene.

Meanwhile, autoimmune diseases are involved with T-cell activation. As a therapeutic gene for the autoimmune diseases, a gene capable of fundamentally inhibiting the activation of T-cells can be considered.

For the activation of T-cells, two signals provided by an antigen-presenting cell (APC) are generally required. The first signal is mediated via the T cell receptor/CD3 complex and an antigen presented by a major histocompatibility complex (MHC) molecule of APC. Such the signal induces the activation of specific T-cells that recognize the MHC/antigen complex. The second signal is also known as a costimulation signal and induces proliferation of T cells. This signal is mediated via B7 of APC. B7 is the counter-receptor which has two ligands, CD28 and CTLA-4, expressed on T lymphocytes. The first ligand, termed CD28, is constitutively expressed on T-cell membrane, and after ligation to B7, induces IL-2 secretion and proliferation of T-cells. The second ligand, termed CTLA4, is homologous to CD28 and expressed after the activation of T-cells. The affinity of B7 for CTLA4 is 20 times higher than that for CD28, thereby the CTLA4 suppress the activation of T-cells. Thus, the CTLA4 protein can be the most effective reagent to inhibit the B7:CD28 costimulatory pathway.

### DISCLOSURE OF THE INVENTION

Accordingly, in order to use CTLA4 as a therapeutic gene, the present inventors have constructed a therapeutic gene in which the extracellular domain (B7 binding domain) of CTLA4 is bound to a portion of immunoglobulin making the CTLA4 dimeric and at the same time, prolonging the half-life of the protein after in vivo administration. And the present inventors have found that the use of the inventive peptide vector containing the constructed gene showed a therapeutic effect against autoimmune diseases, and reached the present invention.

Therefore, in one aspect, the present invention provides a recombinant peptide vector comprising a leader peptide, linker DNAs and a DNA construct formed by operably linking expression control sequences with a therapeutic gene encoding a fusion protein where the extracellular domain of CTLA4 is bound to the Fc fragment of immunoglobulin, wherein the leader peptide is linked to both ends of the DNA construct by the linker DNAs.

In another aspect, the present invention provides a method for the preparation of a recombinant peptide vector, which comprises the following steps: (1) linking a gene encoding the extracellular domain of CTLA4 with a gene encoding the Fc fragment of immunoglobulin so as to prepare a therapeutic gene; (2) operably linking the therapeutic gene with expression control sequences so as to prepare a DNA construct; (3) synthesizing a leader peptide and linker DNAs and then linking the leader peptide and the linker DNAs together, so as to prepare a peptide vector; and (4) linking the both ends of the DNA construct obtained in the step (2) to the leader peptide by the linker DNAs.

In still another aspect, the present invention provides a composition for the treatment of autoimmune diseases, which comprises a pharmaceutically effective amount of the recombinant peptide vector and a pharmaceutically acceptable carrier.

As used herein, the term “autoimmune diseases” means the diseases caused by an immune response to a self-antigen in the body. The autoimmune diseases are the diseases occurring throughout the entire system of the human body, and examples thereof include skin diseases, such as psoriasis, atopic dermatitis, aphthous stomatitist, and systemic lupus erythematosus, type 1 diabetes (also called juvenile diabetes), endocrine system diseases, such as chronic thyroiditis, hematopoietic system diseases, such as aplastic diseases, digestive system diseases, such as hepatitis, primary cirrhosis, ulcerative colitis or Crohn's disease, respiratory system diseases, such as asthma, silicosis or asbestosis, and kidney diseases, such as immunoglobulin kidney disease or post streptococcal glomerulonephritis. In addition, all diseases which will be recognized as autoimmune diseases later are included in the diseases subject to the treatment of this invention.

I. Therapeutic Gene and Peptide Vector

As used herein, the term “therapeutic gene” means a gene which is administered for the complete recovery, inhibition and alleviation of autoimmune diseases. Concretely, the term “therapeutic gene” refers to a gene encoding a fusion protein (also referred to as CTLA4-Ig) formed by binding the extracellular domain of CTLA4 to the hinge of an immunoglobulin Fc fragment.

As used herein, the term “extracellular domain” indicates a domain exposed extracellularly with respect to a transmembrane domain composed of hydrophobic amino acids in a membrane protein located in a cell membrane consisting of phospholipid. This domain consists mainly of hydrophilic amino acids, and is folded toward the protein surface, thereby being soluble in aqueous solution. In most of cell surface receptor proteins, the extracellular domain performs the function of binding to ligands, and the intracellular domain performs the function of intracellular signal transduction.

As used herein, the term “immunoglobulin” means a protein molecule which is produced by B-cells and acts as a antigen receptor of B-cell, so as to specifically recognize a variety of antigens. This molecule has a Y-shaped structure and is composed of two identical light chains and two identical heavy chains. Both the light and heavy chains contain variable and constant regions. The four chains are held together by disulfide bonds located at a flexible hinge domain of heavy chains. The variable regions in both the heavy and light chains are bound to each other to form two identical antigen-binding domains. Immunoglobulins are divided into five classes, A(IgA), D(IgD), E(IgE), G(IgG) and M(IgM), based on their heavy chain constant regions. The five classes are called isotypes and have unique structural characteristics and different biological properties. For example, IgG has a slight difference in the Fc structure from other isotypes. Also, IgG and IgA are divided into many subclasses. For example, human IgG isotype is divided into four subclasses, IgG1, IgG2, IgG3 and IgG4, which have γ1, γ2, γ3 and γ4 heavy chains, respectively. The functions of the immunoglobulin molecule, such as complement activation, binding to phagocyte-Fc receptors, and antigen-dependent cytotoxity, are mediated by structural determinants present in Fc fragment of a heavy-chain. This Fc fragment of a heavy-chain is used as the component of the recombinant peptide vector according to the present invention and can be derived from immunoglobulins of all the above-described classes or subclasses.

As used herein, the term “immunoglobulin Fc fragment” indicates a fragment among the functionally divided fragments of immunoglobulin, which has no antigen-binding ability but is easily crystallized, and is formed by the binding the hinge region and the CH2 and CH3 domains, and is parts involved in binding to effective substances and cells in antibodies. The Fc fragment mentioned in connection with the present invention may be different from those described in some literatures but contains the hinge region, and this term is used merely for the convenience of description of the present invention and will be sufficiently understood by any person skilled in the art with reference to the specification of the present invention and the accompanying drawing.

Meanwhile, as well known in the art, in order to increase the expression level of a therapeutic gene in cells (or target cells) to be introduced with the recombinant peptide vector, the therapeutic gene must be operably linked to transcriptional and translational expression control sequences which are functional in the target cells. Particularly, since the peptide vector which is characteristically used in the present invention contains no separate control sequences, it is preferable that the therapeutic gene should be operably linked to the expression control sequences so as to form a “DNA construct which is then introduced into a peptide vector. In another embodiment, the inventive recombinant peptide vector may also be prepared by linking the expression control sequences with the peptide vector and then introducing the therapeutic gene into the resulting peptide vector structure. As used herein, the term “DNA construct” means a DNA product formed by operably linking the expression control sequences with the therapeutic gene according to the present invention.

As used herein, the term “expression control sequences” refers to nucleic acid sequences necessary or beneficial for the expression of the inventive therapeutic gene. Each of the expression control sequences may be native or foreign to a nucleic acid sequence encoding a fusion protein. Such expression control sequences include, but are not limited to, leader sequences, polyadenylation sequences, propeptide sequences, promoter, enhancer or upstream activation sequences, signal peptide sequences and transcription terminators. In the present invention, the expression control sequences preferably include promoters, signal peptide sequences, and polyadenylation sequences. Other control sequences may be optionally included to further increase the expression level of the inventive therapeutic gene.

For the expression of the inventive therapeutic gene, any expression control sequences may be used if it is suitable to direct expression in mammalian cells. Examples of such control sequences include the early and late promoters of SV40 and adenovirus (e.g., adenovirus major late promoters), MT-1 (metallothionein gene) promoters, human cytomegalovirus (CMV) early genes, human elongation factor 1α, (EF-1α), drosophila minimal heat shock protein 70 promoters, Rous sarcoma virus (RSV) promoters, human ubiquitin C (UbC) promoters, human growth hormone transcription terminators, adenovirus E1b region polyadenylation sequences and bovine growth hormone (BGH) polyadenylation sequences.

As used herein, the term “signal peptide sequence” refers to an amino acid sequence that induces an expressed protein to be transported outside of a cell membrane. Generally, the surface or secretion protein which is transported outside of the cell membrane has an N-terminal sequence which is cut with signal peptidase in the cell membrane. As the signal peptide sequence in the present invention, any sequence suitable to direct secretion in mammalian cells may be used. Preferably, a signal peptide sequence derived from a secretion protein of the same or related species. A signal peptide sequence of hG-CGF, murine immunoglobulin kappa light chains or human oncostatin M may be used but is not limited thereto.

As used herein, the term “operably linked” means a condition where nucleic acids are placed into a functional relationship with another nucleic sequence. This may be a gene and control sequence(s) which are linked to each other in such a manner that the expression of the gene becomes possible when a suitable molecule (e.g., transcription activator protein) is bound to the control sequence(s). For example, if a promoter controlled the transcription of coding sequences, the promoter would be operably linked to the sequences. Generally, the term “operably linked” means that the DNA sequences being linked are contiguous, and, in the case of a secretory leader, contiguous and in reading frame. The linking of such sequences is performed by ligation at suitable restriction enzyme sites. If such sites do not exist, the synthetic oligonucleotide adaptor or linker according to a conventional method may be used.

Meanwhile, as used herein, the term “vector” means a carrier capable of stably carrying a therapeutic gene into target cells. The term “peptide vector” includes a vector disclosed in Korean Patent Laid-Open Publication No. 10-2001-0053621 (entitled “peptide vector”) filed by the present inventors, and consists of a complex of peptide and DNA. The “recombinant peptide vector” means a peptide vector comprising a DNA construct formed by operably linking expression control sequences with a therapeutic gene so that the therapeutic gene can be expressed in target cells.

In the present invention, the peptide vector which is used to carry the therapeutic gene into cells generally consists of (1) a leader peptide and (2) a linker DNAs.

The leader peptide acts to enter cells through cell membranes. Concretely, the leader peptide may have an acetyl group attached to N-terminal amine group in order to eliminate reactivity with other molecules, and may have Cys at the C-terminal end in order to bind the linker DNAs with a disulfide bond.

According to the present invention, the peptide vector may comprise a leader peptide consisting of 16 amino acids. The inventive leader peptide consisting of 16 amino acids may be provided in various embodiments. The 1^{st} to 4^{th} amino acids act to easily penetrate into the phospholipid of a cell membrane and can be selected from amino acids with non-polar aliphatic side chains, for example, Gly, Als, Val, Leu, and ILe. The 5^{th} and 6^{th} amino acids act as a support of maintaining the four amino acids at a stable penetration state upon penetration into the cell membrane, and can be selected from amino acids with nonionic polar side chains, for example, Asn, Gln, Ser, and Tyr. The 7^{th} amino acid acts as a spacer between the above-described 6 amino acids and the 9 amino acids to be followed, and although it may be any amino acid other than acidic amino acids (Asp, and Glu), Gly is particularly preferred. The 8^{th} to 12^{th} amino acids act to provide attraction that they can be driven into cells by interaction with negative charges inside of cell membrane, and they may be selected from amino acids with basic side chains, for example, Lys, Arg, and His. The 13^{th} amino acid acts as a spacer in view of intervals of the cell membrane, thus may be any amino acid other than acidic amino acids, but Gly is preferred. The 14^{th} and 15^{th} amino acids may be any amino acids.

In a preferred embodiment, the leader peptide may have the following amino acid sequence:

(SEQ ID NO: 21)

AC-Gly-Leu-Gly-Ile-Ser-Tyr-Gly-Arg-Lys-Lys-Arg-
Arg-Gly-Arg-Arg-Cys

The leader peptide sequences corresponding to the amino acid sequence of SEQ ID NO: 21 include all amino acid sequences where (1) the 2^{nd} Leu is substituted with lie, (2) the 4^{th} Ile with Leu, (3) 10^{th} Lys with Arg, (4) the 11^{th} Arg with Ile, or (5) the 13^{th} Gly with Leu, Ile, Arg, or Gln.

Acting as mediators of linking the leader peptide to the inventive DNA construct, the linker DNAs may consist of 15-18 nucleotides and have various base sequences. In a preferred embodiment, the linker DNAs may have the following base sequences:

| | |
|---|---|
| Linker-1 DNA: | |
| 5′-Cys-CTA-ATA-CGA-CTC-ACT-AT-3′ | (SEQ ID NO: 22) |
| Linker-2 DNA: | |
| 3′-GAT-TAT-GCT-GAG-TGA-T-\textcircled{p}-5′ | (SEQ ID NO: 23) |

The formation of the peptide vector, i.e., the linking of the leader peptide to the linker DNAs, is achieved by a disulfide bond between the C-terminal Cys of the leader peptide and the 5′-terminal Cys of the linker-1 DNA. In this case, the linker-2 DNA and the leader peptide are not linked to each other by any covalent bond, but since the linker-2 DNA and the linker-1 DNA are annealed together by a complementary base sequence, the leader peptide and the linker-DNAs (both the linker-1 DNA and the linker-2 DNA) may be linked to each other. In such a linked state, a nick is present between the leader peptide and the linker-2 DNA.

Subsequently, the formation of a recombinant peptide vector, particularly the linking of the peptide vector to the inventive DNA construct from which 5′-terminal phosphate group is removed, is achieved by a phosphodiester bond between the 5-terminal phosphate group ( [symbol] ) of the linker-2 DNA of the peptide vector and the 3′-terminal hydroxyl group (OH) of the DNA construct. In this regard, the linker-2 DNA and the leader peptide are not linked by any covalent bond, but since the linker-2 DNA and the linker-1 DNA are annealed together by a complementary base sequence, the leader peptide and the linker DNAs (both the linker-1 DNA and the linker-2 DNA) may be linked to each other. In such a linked state, a nick is present between the leader peptide and the linker-2 DNA. In addition, since the phosphodiester bond is formed at both ends of the DNA construct, a recombinant peptide vector where the leader peptide is linked to both ends of the DNA construct by the linker DNAs is ultimately completed.

The recombinant peptide vector prepared by this specific linking allows easy separation of the inventive DNA construct from the peptide vector when it was introduced into the nuclei of cells.

II. Preparation Method

In another aspect, the present invention provides a method for preparing a recombinant peptide vector, which comprises the following steps: (1) linking a gene encoding the extracellular domain of CTLA4 with a gene encoding the Fc fragment of immunoglobulin so as to prepare a therapeutic gene; (2) operably linking the therapeutic gene with expression control sequences so as to prepare a DNA construct; (3) synthesizing a leader peptide and linker DNAs and then linking the leader peptide and the linker DNAs together, so as to prepare a peptide vector; and (4) linking the both ends of the DNA construct obtained in the step (2) to the leader peptide by the linker DNAs.

In the preparation method according to the present invention, the steps (1), (2) and (3) may be performed independently in any order. In addition, as described above, the preparation may also be performed by linking the expression control sequences to the peptide vector and then linking the therapeutic gene to the resulting peptide vector structure.

Hereinafter, each step of the method for preparing the recombinant peptide vector will be described.

(1) Step of Preparing Therapeutic Gene

The therapeutic gene may be prepared by the following steps: (a) preparing each of a gene encoding the extracellular domain of CTLA4 and a gene encoding the Fc fragment of immunoglobulin, (b) inserting the same restriction enzyme recognition sequences into the prepared gene encoding the extracellular domain of CTLA4 and the prepared gene encoding the Fc fragment of immunoglobulin, by PCR; (c) cutting the restriction enzyme recognition sequences of the CTLA4 extracellular domain-encoding gene and the immunoglobulin Fc fragment-encoding gene with restriction enzymes corresponding to the restriction enzyme recognition sequences; and (d) linking the cut parts of the two DNAs together by ligase, thus preparing a therapeutic gene where the extracellular domain of CTLA4 is bound to the Fc fragment of immunoglobulin.

In one embodiment, the gene encoding the extracellular domain of CTLA4 and the gene encoding the Fc fragment of immunoglobulin may be easily obtained by performing RT-PCR on template mRNA isolated from the cells of species (preferably individual) to be treated, using oligonucleotide primers encoding both ends of the CTLA4 extracellular domain-encoding gene or the immunoglobulin Fc fragment-encoding gene.

In another embodiment, such genes may also be synthesized by any standard method known in the art, e.g., by use of an automatic DNA synthesizer (commercially available from Biosearch, Applied Biosystems, etc). For example, phosphorothioate oligonucleotide may be synthesized by the method described in Stein et al., Nucl. Acids Res. 16:3209 (1988). Methylphosphonate oligonucleotide may be prepared by use of a controlled pore glass polymer support (Sarin et al., Proc. Natl. Acad. Sci. U.S.A. 85:7448-7451 (1988)).

(2) Step of Preparing Inventive DNA Construct

It is preferable in the present invention that the prepared therapeutic gene is operably linked to expression control sequences so as to form a DNA construct, and that the DNA construct is introduced into a peptide vector. The linking between these sequences is performed by ligation at suitable restriction enzyme sites. If such sites do not exist, an oligonucleotide adaptor or linker synthesized according to a conventional method may be used. In order to operably link, it is desirable to link the DNA fragments in view of a specific order and orientation.

DNA may be cut with a certain restriction enzyme in a suitable buffer. Generally, about 0.2-1 μg of a DNA fragment is used with about 1-2 units of the corresponding restriction enzyme in about 20 μl of buffer solution. Suitable buffer, DNA concentration, culture time and temperature can be specified by restriction enzyme manufacturers. It is generally suitable to culture at 37° C. for about 1-2 hours, but some enzymes require a higher temperature. After culture, enzymes and other impurities are removed by extracting the digestive solution with a mixture of phenol and chloroform, and the digested DNA is precipitated with ethanol and collected from the aqueous layer.

The cut DNA fragments are separated and selected according to size by electrophoresis. The DNA can be electrophoresised on agarose or polyacrylamide matrix. The selection of a matrix depends on the size of the DNA fragment to be separated. After electrophoresis, DNA is extracted from the matrix by electroelution, or when low-melting agarose was used, the agarose is molten from which DNA is extracted.

DNA fragments to be linked together are added to a solution at the same molar amount. Such a solution generally contains ATP, ligase buffer, and ligation enzyme, such as T4 ligase of about 10 units per 0.5 μg of DNA. For the linking of the DNA fragments to a vector, the vector may be linearized by cutting with a suitable restriction enzyme and then treated with alkaline phosphatase or bovine intestinal hydrolase. This phosphatase treatment prevents the self-ligation of the vector during the linking process.

In one embodiment of a method of preparing the inventive DNA construct, the inventive therapeutic gene is operably linked to the expression control sequence of an expression vector, which is known to effectively direct expression in mammals. Then, the linked gene structure is cut with a suitable restriction enzyme in such a manner that the cut fragment contains the therapeutic gene and the expression control sequences operably linked to the therapeutic gene. In this way, the DNA construct according to the present invention may be easily obtained.

(3) Step of Preparing Peptide Vector

The leader peptide, a component of the inventive peptide vector, may be easily prepared by chemical synthesis methods generally known to persons skilled in the biochemical field (Creighton, Proteins: Structures and Molecular Principles. W.H. Freeman and Co., NY (1983)). Typical methods include, but are not limited to, liquid or solid phase synthesis, fragment condensation, and F-MOC or T-BOC chemistry (Chemical Approaches to the Synthesis of Peptides and Proteins, Williams et al. Eds., CRC Press, Boca Raton Fla., (1997); A Practical Approach Atherton & Sheppard, Eds., IRL Press, Oxford, England, (1989)).

According to a conventional solid phase method, the leader peptide under the present invention may be synthesized by the condensation reaction between protected amino acids in the order of the C-terminal end, the first amino acid, the second amino acid, the third amino acid, etc. After the condensation reaction, a protective group and a carrier linked to the C-terminal amino acid may be removed by a known method, such as acid decomposition or aminolysis. The above-described peptide synthesis method is described in the literatures (Gross and Meienhofer's. The Peptides, vol 2., Academic Press (1980)).

Solid phase carriers which can be used for the synthesis of the peptide according to the present invention are carriers generally used in the biochemical field, and typical examples thereof include, but are not limited to, polystyrene resins of substituted benzyl type, polystyrene resins of hydroxymethylphenylacetic amide type, substituted benzhydrylpolystyrene resins, and polyacrylamide resins with functional groups capable of binding to peptides, etc.

The protective groups of the initial protected amino acid are used in the conventional peptide synthesis and easily removed by a conventional method, such as acid decomposition, reduction or aminolysis. Specific examples of the amino protective groups include formyl; trifluoroacetyl; benzyloxycarbonyl; substituted benzyloxycarbonyl such as ortho- or para-chlorobenzyloxycarbonyl and ortho- or para-bromobenzyloxycarbonyl; and aliphatic oxycarbonyl such as t-butoxycarbonyl and t-amyloxycarbonyl. The carboxyl groups of amino acids can be protected by conversion into ester groups. The ester groups include benzyl ester, substituted benzyl ester such as methoxybenzyl ester, and alkyl ester such as cyclohexyl ester, cycloheptyl ester or t-butyl ester. Guanidino groups require no protective groups, but may be protected with nitro, tosyl, or arylsulfonyl such as methoxybenzenesulfonyl or mesitylenesulfonyl. Protective groups of imidazole include tosyl, benzyl and dinitrophenyl etc. The indole groups of tryptophan do not require protective groups but may be protected with a protective group such as formyl.

The separation of the peptide from the protective groups and the carrier may be performed by anhydrous hydrofluoride in the presence of various scavengers. Examples of the scavengers include anisole, ortho-, meta- or para-cresol, dimethylsulfide, thiocresol, ethanediol, and mercaptopyridine, which are conventionally used in peptide synthesis.

The linker DNAs which are other components of the inventive peptide vector may be synthesized by any standard method known in the art, for example, by use of an automatic DNA synthesizer (e.g, commercially available from Biosearch, Applied Biosystems, etc.). For example, phosphorothioate oligonucleotide may be synthesized by the method described in Stein et al., Nucl. Acids Res. 16:3209 (1988). Methylphosphonate oligonucleotide may be prepared by use of a controlled pore glass polymer support (Sarin et al., Proc. Natl. Acad. Sci. U.S.A. 85:7448-7451 (1988)).

The leader peptide and linker DNAs so prepared may be linked together by linking the leader peptide to the linker-1 DNA by a disulfide bond, adding the linker-2 DNA to the linked structure, and annealing the linker-1 DNA with the linker-2 DNA by a base pair. In this case, the disulfide bond between the leader peptide and the linker DNAs can be formed in an alkaline condition of about pH 10, to which an antioxidant such as DTT (dithiothreitol) is preferably added. This is because the antioxidant acts to prevent the oxidation of proteins and to protect the activity of enzymes, etc., and can protect the formed disulfide bond and maintain the activity of enzymes upon subsequent ligation. Then, the annealing between the linker-1 DNA and the linker-2 DNA may be achieved by maintaining them at 50° C., a temperature suitable for annealing.

(4) Step of Ligating Inventive DNA Construct with Peptide Vector

Before linking the inventive DNA construct prepared in the step (1) with the peptide vector prepared in the step (2), the DNA construct is preferably treated with phosphatase to remove the 5′-terminal phosphate group. This makes the DNA construct cannot react or bind with the 3′-terminal hydroxyl group of the linker-1 DNA. When the DNA construct from which the phosphate group is removed is mixed with the peptide vector, and a suitable ligase is added to the mixture, the DNA construct and the linker DNAs will be linked together by a phosphodiester bond between the 5′-terminal phosphate group of the linker-2 DNA and the 3′-terminal hydroxyl group of the DNA construct. In this case, since the 3′-terminal hydroxyl group is located at both ends of the DNA construct, the leader peptide will be ultimately linked by the linker DNAs to both ends of the DNA construct.

III. Pharmaceutical Composition

The present invention provides a composition for the treatment of autoimmune diseases, which comprises a therapeutically effective amount of the recombinant peptide vector, and a pharmaceutically acceptable carrier.

The pharmaceutically acceptable carrier contained in the inventive composition is conventionally used in formulation, and examples thereof include, but are not limited to, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methylhydroxy benzoate, propylhydroxy benzoate, talc, magnesium stearate, and mineral oil. The inventive composition may additionally comprise lubricant, wetting agent, sweetening agent, flavors, emulsifier, suspending agent, and preservatives, etc.

The inventive pharmaceutical composition may be administered by routes which are conventionally used in gene therapy. Preferably, it may be administered by parenteral routes, e.g., intravenous, intraabdominal, intramuscular, subcutaneous or local administration routes.

A suitable dose of the inventive pharmaceutical composition may vary depending on many factors, such as formulation methods, administration modes, the age, body weight, sex, disease severity, diet, excretion and response sensitivity of patients, as well as administration time and administration route. An ordinarily skilled physician may easily determine and prescribe a dose effective for the desired treatment. For example, the recombinant peptide vector in the inventive pharmaceutical composition may be administered at a dose of 1 μg/kg/day to 100 μg/kg/day.

The pharmaceutical composition containing the inventive recombinant peptide vector may be formulated with pharmaceutically acceptable carriers and/or excipients into either unit-dose forms or products in multidose containers, according to methods which can be easily performed by persons skilled in the art of the present invention. The resulting formulations may be in the form of a solution, suspension or emulsion in oil or aqueous medium, as well as extracts, powders, granules, tablets or capsules, and may additionally comprise dispersing agents or stabilizers.

The pharmaceutical composition containing the inventive recombinant peptide vector may be freeze-dried in order to increase stability at room temperature, reduce a need for expensive low-temperature storage, and extend shelf life. The freeze-drying process may comprise continuous steps consisting of freezing, first drying and second drying. The second drying step after freeze-drying the composition comprises lowering pressure and heating for the sublimation of vapor. The second step is performed to evaporate the remaining absorbed moisture from dried materials.

In one embodiment, the freeze drying of the inventive pharmaceutical composition is performed in the following order: (1) the collapse temperature of the formulation is determined by use of freeze-drying microscopy (Pikal, M. J. et al. Int. J. Pharm., 1990, vol. 62, p. 165); (2) a vial is placed on a freeze-dryer shelf under room temperature and then equilibrated at −1° C. for about 30 minutes; (3) the shelf is cooled to −55° C. and maintained at this temperature for 2 hours; (4) second drying is performed at a product temperature of about −32° C. or a 5° C. lower temperature than the collapse temperature; (5) second drying is performed at 35° C. Chamber pressure is controlled to 55-120 mmHg and then the drying is completed; (6) the vial is plugged under the vacuum of the freeze-dryer, and the freeze-dried vial is crimp-sealed and stored at 2-8° C.

The freeze-dried formulation may contain excipients and lyoprotectant. The excipients include, but are not limited to, buffers containing 0.9% NaCl and 10 mM sodium phosphate (pH 7.0) or 10 mM sodium citrate (pH 7.0). The lyoprotectant acts to protect biological molecules during freezing and drying steps and to impart a mechanical support to final products, and examples thereof include PBS (pH 7.0). PBS/4%, 12% or 15% trehalose, etc.

### BRIEF DESCRIPTION OF THE DRAWINGS

FIG. 1 is a schematic diagram of a recombinant peptide vector according to the present invention. CMV: a cytomegalovirus promoter; SP: an oncostatin M signal peptide sequence; V: the extracellular domain of CTLA4; H, CH2 and CH3: the Fc fragments of IgA; BGH: bovine growth hormone polyadenylation sequence; and alphabets at the bottom of the figure: the hinge (H) domain amino acid sequence of IgA where the symbol * denotes a cysteine-to-serine substitution.

FIG. 2(a) shows the results of RT-PCR and electrophoresis performed for RNAs extracted from various tissues of test group rats administered with the inventive recombinant peptide vector and control group rats administered with no inventive recombinant peptide vector, in order to examine whether a therapeutic gene is expressed in a host. M: 100-bp ladder; lanes 1 and 6: liver; lanes 2 and 7: kidney; lanes 3 and 8: spleen; lanes 4 and 9: lungs; lanes 5 and 10: muscles; and lanes 11 and 12: negative control group administered with distilled water. FIG. 2(b) shows the results of RT-PCR and electrophoresis performed for RNA extracted from the blood of test group dogs administered with the inventive recombinant peptide vector and control group dogs administered with no inventive recombinant peptide vector. Lane N: negative control dogs administered with distilled water; lane C: negative control dogs administered with no recombinant peptide vector; lanes 0, 1, 3, 7, 11, 15, 19, 26 and 30: results for dogs administered with a recombinant peptide vector, at 0, 1, 3, 7, 11, 15, 19, 26 and 30 days after administration with the recombinant peptide vector, respectively; and lanes 21 and 168: results for other dogs administered with a recombinant peptide vector, at 21 and 168 days after administration with the recombinant peptide vector, respectively.

FIG. 3 graphically shows the results of measurement of an antibody to a recombinant peptide vector in test group dogs (dog 1 and dog 2) administered with a recombinant peptide vector and control dogs with administered with no recombinant peptide vector, in which the antibody measurement was performed at 0, 3, 7, 15 and 30 days after gene treatment in order to examine whether the antibody to the recombinant peptide vector is produced.

FIG. 4 is a photograph showing hair loss conditions before and after gene treatment with the inventive recombinant peptide vector. (a), (c), (e) and (g): before gene treatment; and (b), (d), (f) and (h): after gene treatment.

FIG. 5 shows the results of H & E staining performed in order to examine the conditions of skin tissue before and after gene treatment with the inventive recombinant peptide vector. (a) and (b): the amounts of lymphocytes and plasma cells penetrated into the upper dermal layer before and after gene treatment with the inventive recombinant peptide vector, respectively; (c) and (d): the conditions of follicles before and after gene treatment, respectively; and (d) and (e): the amount of immunoglobulin deposited on the dermis-epidermis junction before and after gene treatment, respectively.

### BEST MODE FOR CARRYING OUT THE INVENTION

Hereinafter, the present invention will be described in more detail by the following examples. It will however be obvious to a person skilled in the art that the scope of the present invention is not limited to or by these examples.

### Example 1

(1) Amplification of Coding Sequence of Dog CTLA4

1.4 ml of CPDA (citrate phosphate dextrose acid) as an anticoagulant was added to 10 ml of venous blood taken from a healthy dog and then subjected to Ficoll-Plaque gradient centrifugation to isolate peripheral blood mononuclear cells (PBMCs). The isolated PBMCs were washed two times with PBS (phosphate-buffered saline), adjusted to 1×10⁶ cells/ml in complete endotoxin-free medium RPMI 1640 (containing 10% fetal calf serum, 50 μl/ml gentamicin, 10 μl/ml concanavaline A), and cultured under 5% CO₂ at 37° C. for 4 hours. After the culture, PBMCs were collected by centrifugation and frozen in liquid nitrogen.

From the isolated PBMCs, total RNA was separated using a Trizol reagent and washed with 75% ethanol, and the RNA pellets were dissolved in DEPC-treated water. 2 μg of the purified mRNA and 1 μl of an oligo (dT) 30 primer were mixed, heated at 70° C. for 2 minutes and cooled by addition of ice. To the mixture, 200 U M-Mulv reverse-transcriptase, 5 μl of five-fold buffer, 1 μl of dNTP and DEPC-treated water were added to a total volume of 50 μl. The mixture was allowed to react at 42° C. for 1 hour so as to synthesize primary cDNA.

The primary cDNA template obtained as described above was subjected to PCR with the following primer pair, so as to amplify 758-bp CTLA4. Specifically, the PCR reaction was performed after adding 3 μl of primary cDNA, 2 U Pfu DNA polymerase, 10 μl of 10-fold buffer, 1% Triton X-100, 1 mg/ml bovine serum albumin (BSA), 3 μl of CTLA4 forward primer (10 μM), 3 μl of CTLA4 reverse primer (10 μM), 2 μl of dNTP (each 10 mM) and triple distilled water to a total volume of 100 μl. Also, the PCR reaction consisted of the following: 3 min at 95° C.; 30 cycles of 30 sec at 95° C., 1 min at 52° C., and 1 min and 30 sec at 72° C.; and 10 min at 72° C. The PCR product had completely blunt ends.

| | |
|---|---|
| CTLA4 forward primer: | |
| 5′-AAGACCTGAACACTGCTCCA-3′ | (SEQ ID NO: 1) |
| CTLA4 reverse primer: | |
| 5′-TTGAAATTGCCTCAGCTCCT-3′ | (SEQ ID NO: 2) |

(2) Amplification of Coding Sequence of Dog Immunoglobulin (IgA) Fc Fragment

In order to amplify the Fc fragment of IgA (H—CH2-CH3 domain), the PBMCs obtained as described above were activated with 5 μl/ml LPS, and then total RNA was extracted from the cells in the same manner as in the above part (1) and subjected to RT-PCR. The resulting primary cDNA template was subjected to PCR with the following primer pair, thus amplifying a 726-bp IgA Fc fragment.

| | |
|---|---|
| IgA forward primer: | |
| 5′-GATAACAGTCATCCGTGTCA-3′ | (SEQ ID NO: 3) |
| IgA reverse primer: | |
| 5′-GTAGCAGATGCCGTCCACCT-3′. | (SEQ ID NO: 4) |

### Example 2

### Ligation of CTLA4 Extracellular Domain to IgA Fc Fragment

For the ligation between the CTLA4 extracellular domain and the IgA Fc fragment amplified in Example 1, a forward primer (containing a HindIII restriction enzyme recognition sequence) and a reverse primer (containing an Eco RI restriction enzyme recognition sequence) were set for the amplification of the CTLA4 extracellular domain, and likewise, a forward primer (containing an Eco RI restriction enzyme recognition sequence) and a reverse primer (containing a Xba I restriction enzyme recognition sequence), which are the same as used in Example 1 (2), were set for the amplification of the IgA Fc fragment. Then, PCR was performed with such primers.

In this case, since a CTLA4 signal peptide was not identified, the signal peptide of human oncostatin M was attached to the 5′-terminal end of the amplified CTLA4, for extracellular secretion. This was achieved by two-step overlapping PCR. The first-step PCR was performed with the following forward and reverse primers synthesized so that they contain 15 amino acids from the signal peptide of oncostatin M and 7 amino acids at the N-terminal end of CTLA4.

SP-CTLA4 forward primer: 5′-

(SEQ ID NO: 5)

CTCAGTCTGGTCCTTGCACTCCTGTTTCCAAGCATGGCGAGCATGTCCAA
AGGGATGCATGTGGCT-3′

SP-CTLA4 (EcoR I) reverse primer:

| | |
|---|---|
| 5′-GAATTCGTCAGAATCTGGGCAAGGTTC-3′ | (SEQ ID NO: 6) |

The second-step PCR was performed by purifying the PCR product from the first-step PCR and then amplifying the purified product with the following forward primer containing a HindIII restriction enzyme recognition sequence at the N-terminal end of oncostatin M and the SP-CTLA4 (EcoR I) reverse primer as described above.

SP-CTLA4 (HindIII) forward primer:

(SEQ ID NO: 7)

5′-AAGCTTCACCATGGGTGTACTGCTCACACAGAGGACGCTGCTCAGTC
TGGTCCTTGCACTC-3′

The IgA Fc fragment was reamplified with the following primers:

IgA (Eco RI) forward primer:

| | |
|---|---|
| 5′-GAATTCGATAACAGTCATCCGTCTCAT-3′ | (SEQ ID NO: 8) |

IgA (Xba I) reverse primer:

| | |
|---|---|
| 5′-TCTAGAGTAGCAGATGCCGTCCAC-3′ | (SEQ ID NO: 9) |

Using these primer sets, a 472-bp (CTLA4 extracellular domain) PCR product and a 741-bp (IgA Fc fragment) PCR product were obtained. In this process, three cysteins among four cysteins at the IgA hinge domain were substituted with serine so as to form one disulfide bond between chains. Each of the amplified PCR products was cloned into a pCR2.1 vector, so as to prepare pCR2.1-CTLA4 and pCR2.1-IgA. E. coli TOP10 was transformed with these vectors, and positive colonies were selected and cultured in liquid medium. After the culture, plasmids were extracted with the respective restriction enzymes (Hind III and EcoR I for pCR2.1-CTLA4, and EcoR I and Xba I for pCR2.1-IGHAC), and then, the desired bands were separated on agarose gel and ligated to each other. The ligation product was PCR-amplified again and then cloned into a pCR2.1 vector.

### Example 3

### Ligation of Therapeutic CTLA4-Ig Gene to pcDNA 3.1(+)

The therapeutic CTLA4-Ig gene obtained in Example 2 and a pcDNA 3.1(+) vector were treated with HindIII and XbaI, respectively. Then, the therapeutic gene and the vector were separated and purified on agarose gel and ligated. An E. coli TOP10 strain was transformed with this recombinant vector and selectively cultured.

### Example 4

### PCR of Therapeutic Gene

In order to prepare a therapeutic gene which is operably linked to expression control sequences for final use, a DNA construct, the following forward primer was set in front of the CMV promoter of pcDNA3.1(+), and the following reverse primer was set at the back of BGH poly(A) signal:

| | |
|---|---|
| CMV-forward primer: | |
| 5′-GCCAGATATACGCGTTGACAT-3′ | (SEQ ID NO: 10) |
| BGH-reverse primer: | |
| 5′-GCTTAATGCGCCGCTACA-3′ | (SEQ iD NO: 11) |

Using these primers, PCR was performed to obtain a 2213-bp DNA construct with a therapeutic gene operably linked to expression control sequences. The base sequence of this DNA construct is shown in SEQ ID NO: 12.

### Example 5

### Therapeutic CTLA4-Ig Gene Derived from Human Beings

Meanwhile, in order to prepare a therapeutic CTLA4-Ig gene which can be used on human beings, a CTLA4 gene and immunoglobulin gene derived from human beings were used, and all the preparation steps were conducted in the same manner as described in Examples 1 to 4. The base sequence of a primer pair used in each step is as follows, and the resulting DNA construct having a therapeutic gene operably linked to expression control sequences has a base sequence shown in SEQ ID NO: 20.

| | |
|---|---|
| hCTLA4 forward primer: | |
| 5′-AAGACCTGAACACCGCTCCC-3′ | (SEQ ID NO: 13) |
| hCTLA4 reverse primer: | |
| 5′-GTTAGAATTGCCTCAGCTCTT-3′ | (SEQ ID NO: 14) |
| hIgG forward primer: | |
| 5′-GAGCCCAAATCTTGTGACAAAAC-3′ | (SEQ ID NO: 15) |
| hIgG reverse primer: | |
| 5′-AGCATCCTCGTGCGACCGCG-3′ | (SEQ ID NO: 16) |

hSP-CTLA4 forward primer:

(SEQ ID NO: 17)

5′-CTCAGTCTGGTCCTTGCACTCCTGTTTCCAAGCATGGCGAGCATGGC
AATGCACGTGGCCCAGCC-3′

hSP-CTLA4 (EcoR I) reverse primer: the same as the SP-CTLA4 (EcoR I) reverse primer
hSP-CTLA4 (HindIII) forward primer: the same as the SP-CTLA4 (HindIII) forward primer

hIgA (Eco RI) forward primer:

(SEQ ID NO: 18)

5′-GAATTCGAGCCCAAATCTTCTGACAAAACTCACACATCCCCACCGTC
CCCAGCACCTGAACTCCTG-3′

hIgA (Xba I) reverse primer:

(SEQ ID NO: 19)

5′-TCTAGAAGCATCCTCGTGCGACCGCGAGAGC-3′

### Example 6

### Synthesis of Peptide Vector

In the present invention, the peptide vector acting to transduce the therapeutic gene into cells consists of the leader peptide and the linker DNAs.

The leader peptide has the following amino acid sequence, is synthesized by an Fmoc solid-phase method, and contains an acetyl group (Ac) attached to the N-terminal end (Peptron Inc.).

(SEQ ID NO: 21)

Ac-Gly-Leu-Gly-Ile-Ser-Tyr-Gly-Arg-Lys-Lys-Arg-
Arg-Gly-Arg-Arg-Cys

Linker sequence:

(SEQ ID NO: 22)

| | |
|---|---|
| Linker-1 DNA: | 5′-Cys-OO-CTA-ATA-CGA-CTC-ACT-AT-3′ |

wherein —OO-represents an ester bond.

| | |
|---|---|
| Linker-2 DNA: | |
| 3′-GAT TAT GCT GAG TGA-T-5′. | (SEQ ID NO: 23) |

In the linker sequence, for linking with the leader peptide, cystein was bonded to the 5′-terminal end of the linker-1 DNA, and for linking with the therapeutic gene, a phosphate group was attached to the 5′-terminal end of the linker-2 DNA by T4 polykinase (Perkin Elmer).

2 nmol of the leader peptide and 2 nmole of the linker-1 DNA were allowed to react in buffer (containing 50 mM Tris, 0.1 mM EDTA and 10 mM DTT, pH 10.5) at 37° C. for 1 hour, so as to link them together by an S—S bond. Then, for hybridization of the linker-1 DNA with the linker-2 DNA having the phosphate group at the 5′-terminal end, 2 nmol of the linker-2 DNA was added and allowed to react at 60° C. for 30 minutes. Subsequently, 10 pmol (20 pmol/μl) was dispensed each time, and then stored below −20° C.

### Example 7

### Ligation of DNA Construct to Peptide Vector

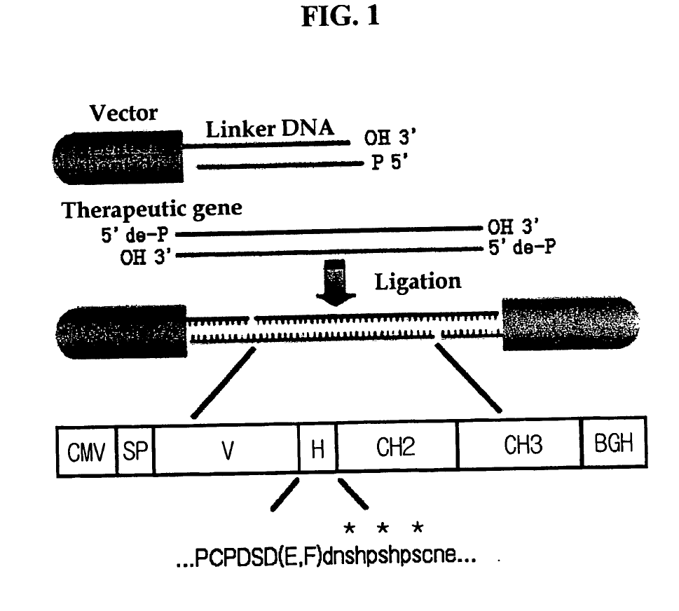

The DNA construct amplified by PCR in Example 5 was purified with silica on an agarose gel fragment, and then ligated to a vector with T4 ligase. After the ligation, the ligated product was confirmed by band shift in agarose gels, and then administered to disease model animals. FIG. 1 shows a schematic diagram of the peptide vector and the DNA construct according to the present invention.

### Example 8

(1) Examination of Delivery of Therapeutic Gene on Rats

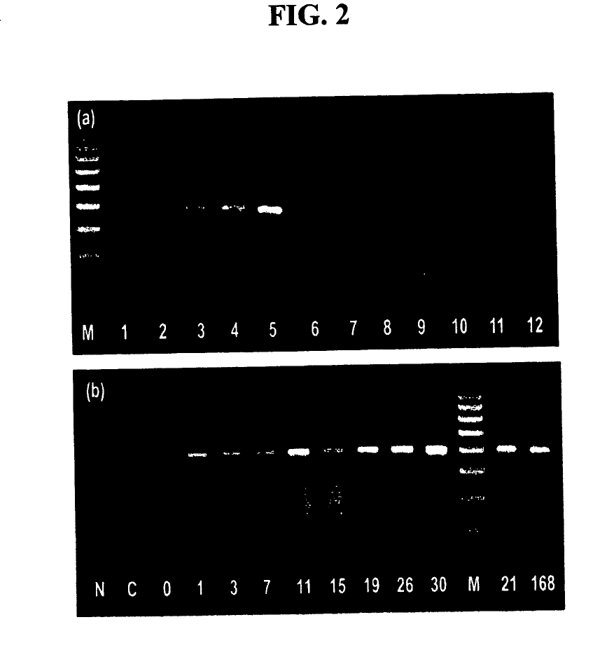

In order to examine whether the peptide vector can deliver the therapeutic gene into various tissues, rats were administered with the recombinant peptide vector, and the RNA of the therapeutic gene was extracted from various tissues of the rats and subjected to RT-PCR. The results are shown in FIG. 2.

FIG. 2a shows the results of RT-PCR and electrophoresis for RNAs extracted from various tissues of test group rats administered with the recombinant peptide vector and control group rats administered with no recombinant peptide vector (M: 100-bp ladder; lanes 1 and 6: liver; lanes 2 and 7: kidney; lanes 3 and 8: spleen; lanes 4 and 9: lungs; lanes 5 and 10: muscles; and lanes 11 and 12: negative control group administered with distilled water). From the results shown in FIG. 2a, it could be found that the therapeutic gene inserted into the recombinant peptide vector was delivered into various tissues, such as liver, kidney, spleen, lungs and muscles, and expressed in the tissues.

(2) Examination of Delivery of Therapeutic Gene on Dogs

In order to examine whether a therapeutic gene is well expressed in dogs, a primer pair capable of amplifying the binding site of therapeutic genes (CTLA4 and IgA) which are not found in normal dogs was set, and using the primer set, RNA taken from the peripheral blood mononuclear cells of dogs was subjected to RT-PCR. The results are shown in FIG. 2b.

FIG. 2b shows the results of RT-PCR and electrophoresis for RNAs extracted from various tissues of test group dogs administered with the inventive recombinant peptide vector and control group dogs administered with no inventive recombinant peptide vector (lane N: a negative control dog administered with distilled water; lane C: a negative control dog administered with no recombinant peptide vector; lanes 0, 1, 3, 7, 11, 15, 19, 26 and 30: test results at 0, 1, 3, 7, 11, 15, 19, 26 and 30 days after administration with the recombinant peptide vector, respectively, in a dog administered with the recombinant peptide vector; and lanes 21 and 168: test results at 21 and 168 days after administration with the recombinant peptide vector, respectively, in another dog administered with the recombinant peptide vector). As shown in FIG. 2b, a 394-bp specific band corresponding to the binding domain of the therapeutic genes (CTLA4 and IgA) was observed, and the therapeutic genes were expressed for at least 168 days after administration of the inventive recombinant peptide vectors.

### Test Example 1

### Measurement of Urine Protein-to-Creatine Ratio

The urine of a dog which had been immunized with heparan sulfate to cause SLE was collected with a catheter between A.M. 10 and P.M. 2. Urine protein was measured by the method described in Lott J A, et al. Clin. Chem. 1983, vol. 29(11), p. 1946, and urine creatine was measured by modified Jaffe reaction after dilution with distilled water at 1:100. A urine protein-to-creatine ratio of less than 0.6 can be evaluated as normal, and a urine protein-to-creatine ratio of more than 1 can be evaluated as severe kidney glomerular diseases (Sodikoff C H. Urine tests. In Sodikoff C H (ed). Laboratory profiles of small animal diseases A guide to laboratory diagnosis. 2nd ed. St. Louis: Mosby, 1995:50). It could be found that the dog, which has showed high urine protein-to-creatin ratio before treatment with the inventive recombinant peptide vector, showed normal urine protein-to-creatin ratio after treatment with the peptide vector. This suggests that the therapeutic gene prepared according to the present invention alleviates kidney glomerular diseases (Table 1).

TABLE 1

| | Before | Days after treatment | | |
|---|---|---|---|---|
| Dogs | treatment | 7 | 32 | 99 |
| Dog 1 | 2.62 | 1.03 | 0.64 | 0.58 |
| Dog 2 | 1.51 | 0.99 | 0.27 | 0.35 |

### Test Example 2

### ELISA for Measurement of Antibody to Peptide Vector

In order to examine whether the peptide vector used in the present invention produces an antibody in a host, competitive ELISA was carried out.

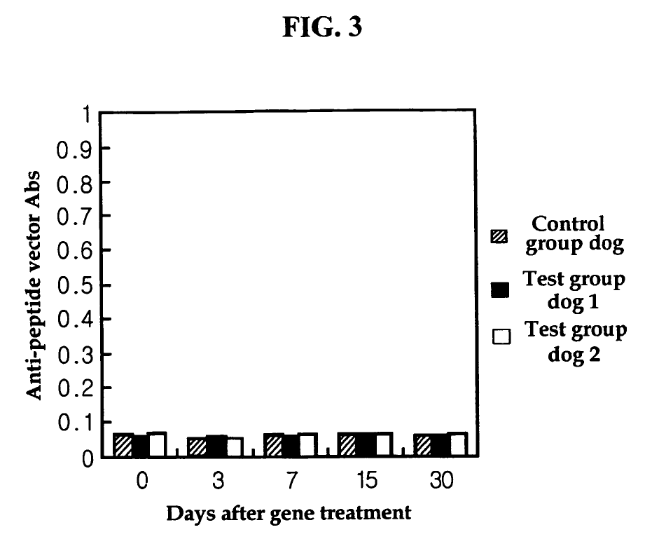

From the control group dogs untreated with the inventive peptide vector and the test group dogs treated with the inventive peptide vector, blood was taken at 0, 3, 7, 15 and 30 days after the gene therapy, and serum was isolated from the blood. 12.5 μg/well of the peptide vector was diluted in PBS and then coated at 4° C. overnight. To block the remaining binding sites, a solution of 1% BSA in PBS was used as blocking buffer. In detection, the dog serum was used at a 1:200 dilution, and as an antibody binding specifically to the peptide vector, a peroxidase-conjugated rabbit anti-dog IgG was used as a secondary antibody, and as a substrate, o-phenylenediamine dihydrochloride (Sigma p9187) was used. 3M HCl as a stop solution was used to stop the reaction, and the absorbance at 492 nm was measured. The results are shown in FIG. 3.

Graphs in FIG. 3 show the comparison of amount of specific antibody bound to the recombinant peptide vector between the test group dogs (dog 1 and dog 2) administered with the recombinant peptide vector and the control dog administered with no peptide vector, in which the antibody amount was performed at 0, 3, 7, 15 and 30 days after the gene therapy. From the results in FIG. 3, it could be found that the antibody to the peptide vector was not produced in the blood of the test group dogs.

### Test Example 3

### Histological Examination

Dogs were administered with heparan sulfate (HS) to cause systemic lupus erythematosus, and after 12 weeks the dog skin tissue was taken. In the case of dogs treated with the inventive recombinant peptide vector, the skin tissue was taken 99 days after the treatment. The taken skin tissue was subjected to H & E (hematoxylin & eosin) staining and immunostaining against immunoglobulin and C3. Heavy chain-specific goat anti-dog IgG, μ chain-specific goat anti-dog IgM and goat anti-dog C3 (benthyl laboratories, Montgomery, Tex.) were diluted at 1:200 and used as primary antibodies, and peroxidase-anti goat IgG (H+L) was diluted at 1:200 and used as a secondary antibody.

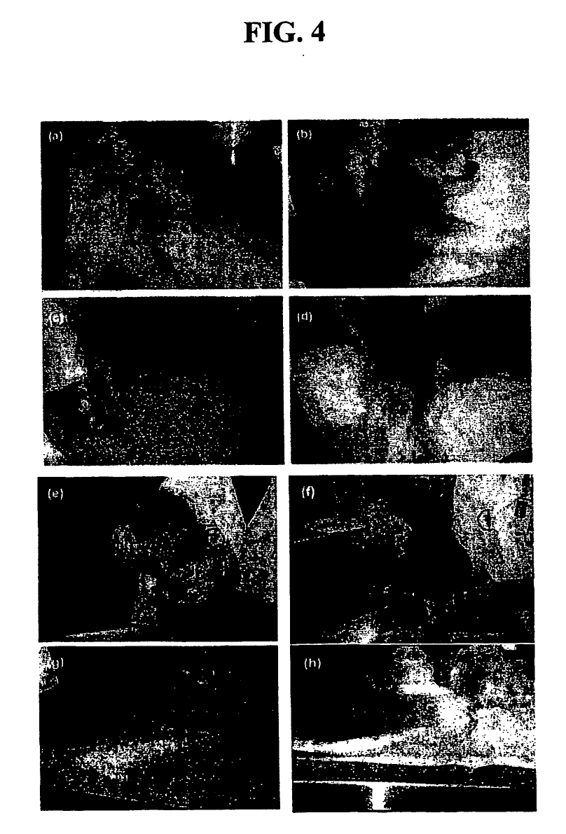

In the case of administration with the therapeutic gene, the result of visual observation showed that systemic lupus erythematosus-related skin diseases, such as alopecia, erythema, boil, scabs and seborrhea, completely disappeared (FIGS. 4a to 4h).

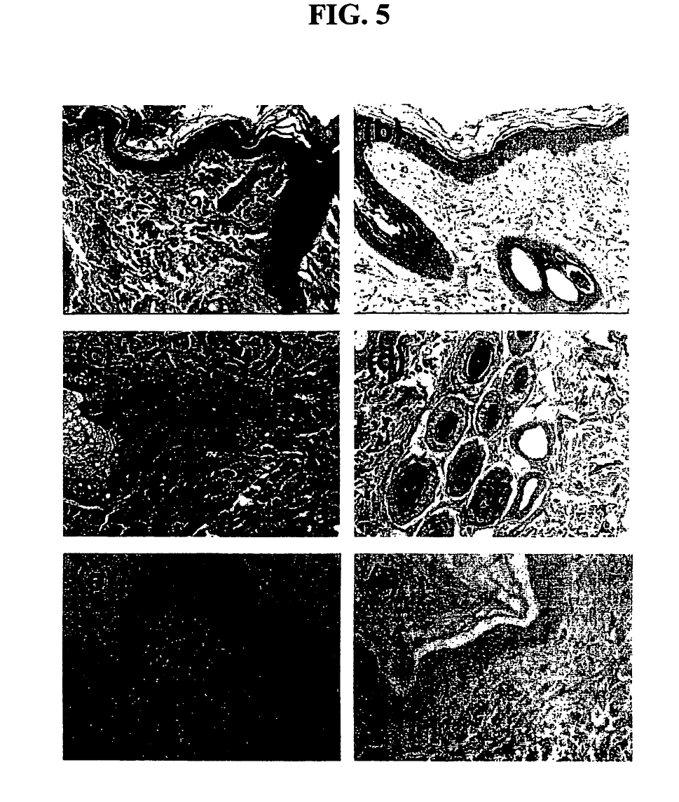

Similarly, the results of H&E staining on the skin tissue taken at 99 days after the gene therapy showed that the number of lymphocytes and plasma cells which had been penetrated into the upper dermal layer was significantly reduced (FIGS. 5a and 5b). Also, fresh hairs were produced in follicles which had been in the resting phase and had no hair, and such follicles were returned to the normal anagenic phase (FIGS. 5c and 5d). Upon the immunostaining on the skin tissue, the penetration of immunoglobulin (IgM) and complements (C3) into the dermis-epidermis junction, also called the lupus-specific band which is characteristically shown in lupus erythematosus, disappeared (FIGS. 5e and 5f).

### Test Example 4

### Examination of Anti-Nuclear Antibody in HS-Immunized Dogs and Gene-Treated Dogs

Using acetone-fixed Crandall-Reese Feline Kidney (CRFK) cells as a substrate, an autoantibody to the nuclei of the cells (anti-nuclear antibody) was measured by indirect immunofluorescence. Dog serum was diluted at 1:2 to 1:128 and measured for fluorescence. The binding to the autoantibody was detected using fluorescein isothiocyanate-conjugated goat anti-dog IgG (1:16 dilution). The fluorescence was observed with an epifluorescence microscope. The serum was taken at 3, 7, 11, 18, 25 and 27 weeks after immunization with HS, and at 7, 32 and 99 days after the gene therapy.

If fluorescence is detected even in serum with high dilution ratio (i.e., the serum shows positive), this means that anti-nuclear antibodies are more present in the serum. Generally, the serum of normal dogs will show fluorescence at dilution ratios up to 1:10. Fluorescence shown at dilution ratios up to 1:16 will be regarded as low-titered positive, and fluorescence at a dilution of 1:64 as high-titered positive. In Table 2 below, the case showing positive even above 1:128 is sufficient to conclusively diagnose as SLE, and the case showing positive only at 1:2 is not different the case of normal animals. Accordingly, it can be found that the recombinant peptide vector effectively treats SLE.

TABLE 2

| | | Days after treatment | | |
|---|---|---|---|---|
| | Before treatment | 7 | 32 | 99 |
| Treated dogs | >1:128 | 1:64 | 1:2 | 1:2 |
| Untreated dogs | >1:128 | >1:128 | >1:128 | >1:128 |

### INDUSTRIAL APPLICABILITY

As described above, by gene therapy using the inventive recombinant peptide vector, the therapeutic gene can be delivered into all cells while minimizing the production of an antibody to the vector. Thus, the inventive recombinant peptide vector will be useful for the treatment of diseases occurring throughout the entire system, particularly such as autoimmune diseases.

1-26. (canceled)

27. A recombinant peptide vector comprising a leader peptide, linker DNAs and a DNA construct formed by operably linking expression control sequences with a therapeutic gene encoding a fusion protein where the extracellular domain of CTLA4 is bound to the Fc fragment of immunoglobulin, wherein the leader peptide is linked to both ends of the DNA construct by the linker DNAs.

28. The recombinant peptide vector of claim 27, wherein the leader peptide consists of 16 amino acids.

29. The recombinant peptide vector of claim 28, wherein the 1^{st} to 4^{th} amino acids are amino acids with non-polar aliphatic side chains.

30. The recombinant peptide vector of claim 29, wherein the amino acids with non-polar aliphatic side chains are selected from the group consisting of Gly, Ala, Val, Leu and Ile.

31. The recombinant peptide vector of claim 28, wherein the 5^{th} and 6^{th} amino acids are amino acids with nonionic polar side chains.

32. The recombinant peptide vector of claim 31, wherein the amino acids with nonionic polar side chains are selected from the group consisting of Asn, Gln, Ser and Thr.

33. The recombinant peptide vector of claim 29, wherein the 7^{th} amino acid is Gly.

34. The recombinant peptide vector of claim 28, wherein the 8^{th} to 12^{th} amino acids are amino acids with basic side chains.

35. The recombinant peptide vector of claim 34, wherein the amino acids with basic side chains are Lys or Arg.

36. The recombinant peptide vector of claim 28, wherein the 13^{th} amino acid is Gly.

37. The recombinant peptide vector of claim 27, wherein the leader peptide has an amino acid sequence of SEQ ID NO: 21.

38. The recombinant peptide vector of claim 27, wherein the linker DNAs have a base sequence formed by annealing a base sequence of SEQ ID NO: 22 with a base sequence of SEQ ID NO: 23.

39. The recombinant peptide vector of claim 27, wherein the DNA construct and one of the linker DNAs are linked together by a phosphodiester bond between the 5′-terminal phosphate group of the one linker DNA and the 3′-terminal hydroxyl group of the therapeutic gene, the leader peptide and the other linker DNA are linked together by a disulfide bond between the C-terminal Cys of the leader peptide and the 5′-terminal Cys of the other linker DNA, and the two linker DNAs are annealed together, thereby linking both ends of the DNA construct to the leader peptide by the linker DNAs.

40. The recombinant peptide vector of claim 27, wherein the CTLA4 and the immunoglobulin are derived from mammals.

41. The recombinant peptide vector of claim 40, wherein the mammals are human beings or dogs.

42. The recombinant peptide vector of claim 27, wherein the expression control sequences include a promoter, a signal peptide sequence and a polyadenylation sequence.

43. The recombinant peptide vector of claim 42, wherein the promoter is a promoter derived from cytomegalovirus.

44. The recombinant peptide vector of claim 42, wherein the signal peptide sequence is a secretory sequence derived from human oncostatin M.

45. The recombinant peptide vector of claim 42, wherein the polyadenylation sequence is derived from bovine growth hormones (BGH).

46. The recombinant peptide vector of claim 27, wherein the DNA construct is a base sequence shown in SEQ ID NO: 12 or SEQ ID NO: 20.

47. The recombinant peptide vector of claim 27, wherein the immunoglobulin is IgA or IgG.

48. A method for preparing a recombinant peptide vector, which comprises the steps of:
(1) linking a gene encoding the extracellular domain of CTLA4 with a gene encoding the Fc fragment of immunoglobulin so as to prepare a therapeutic gene;
(2) operably linking the therapeutic gene with expression control sequences so as to prepare a DNA construct;
(3) synthesizing a leader peptide and linker DNAs and then linking the leader peptide and the linker DNAs together, so as to prepare a peptide vector; and
(4) linking the both ends of the DNA construct obtained in the step (2) to the leader peptide by the linker DNAs.

49. The method of claim 48, wherein the DNA construct and one of the linker DNAs are linked together by a phosphodiester bond between the 5′-terminal phosphate group of the one linker DNA and the 3′-terminal hydroxyl group of the DNA construct, the leader peptide and the other linker DNA are linked together by a disulfide bond between the C-terminal Cys of the leader peptide and the 5′-terminal Cys of the other linker DNA, and the two linker DNAs are annealed together, thereby linking both ends of the therapeutic gene to the leader peptide by the linker DNAs.

50. A composition for the treatment of autoimmune diseases, which comprises a pharmaceutically effective amount of a recombinant peptide vector as claimed in claim 27, and a pharmaceutically acceptable carrier.

51. The composition of claim 50, wherein the autoimmune diseases are systemic lupus erythematosus.


SEQUENCE LISTING (23 sequences)

SEQ ID NO: 1 - DNA, 20 bp, Artificial Sequence
  primer
aagacctgaacactgctcca

SEQ ID NO: 2 - DNA, 20 bp, Artificial Sequence
  primer
ttgaaattgcctcagctcct

SEQ ID NO: 3 - DNA, 20 bp, Artificial Sequence
  primer
gataacagtcatccgtgtca

SEQ ID NO: 4 - DNA, 20 bp, Artificial Sequence
  primer
gtagcagatgccgtccacct

SEQ ID NO: 5 - DNA, 66 bp, Artificial Sequence
  primer
ctcagtctggtccttgcactcctgtttccaagcatggcgagcatgtccaaagggatgcatgtggct

SEQ ID NO: 6 - DNA, 27 bp, Artificial Sequence
  primer
gaattcgtcagaatctgggcaaggttc

SEQ ID NO: 7 - DNA, 61 bp, Artificial Sequence
  primer
aagcttcaccatgggtgtactgctcacacagaggacgctgctcagtctggtccttgcactc

SEQ ID NO: 8 - DNA, 27 bp, Artificial Sequence
  primer
gaattcgataacagtcatccgtctcat

SEQ ID NO: 9 - DNA, 24 bp, Artificial Sequence
  primer
tctagagtagcagatgccgtccac

SEQ ID NO: 10 - DNA, 21 bp, Artificial Sequence
  primer
gccagatatacgcgttgacat

SEQ ID NO: 11 - DNA, 18 bp, Artificial Sequence
  primer
gcttaatgcgccgctaca

SEQ ID NO: 12 - DNA, 2213 bp, Artificial Sequence
  therapeutic gene
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatgggtgtactgctcacacagaggacgctgctcagtctggtccttgcactcctgtttccaagcatggcgagcatgtccaaagggatgcatgtggctcagcctgcagtggttctggccagcagccggggtgttgctagcttcgtgtgtgaatatgggtcttcaggcaacgcagccgaggtccgggtgacagtgctgcggcaggctggcagccagatgactgaagtctgtgccgcgacatacacagtggaggatgagttggccttcctggatgattctacctgcactggcacctccagtggaaacaaagtgaacctcaccatccaagggttgagggccatggacacggggctctacatctgcaaggtggagctcatgtacccaccaccctactatgtaggcatgggaaatggaacccagatttatgtcatcgatcctgaaccttgcccagattctgacgaattcgataacagtcatccgtctcatccatctccctcgtccaatgagccccgcctgtcactacagaagccagccctcgaggatctgcttttaggctccaatgccagcctcacatgcacactgagtggcctgaaagaccccaagggtgccaccttcacctggaacccctccaaagggaaggaacccatccagaagaatcctgagcgtgactcctgtggctgctacagtgtgtccagtgtcctaccaggctgtgctgatccatggaaccatggggacaccttctcctgcacagccacccaccctgaatccaagagcccgatcactgtcagcatcaccaaaaccacagagcacatcccgccccaggtccacctgctgccgccgccgtcggaagagctggccctcaatgagctggtgacactgacgtgcttggtgaggggcttcaaaccaaaagatgtgctcgtacgatggctgcaagggacccaggagctaccccaagagaagtacttgacctgggagcccctgaaggagcctgaccagaccaacatgtttgccgtgaccagcatgctgagggtgacagccgaagactggaagcagggggagaagttctcctgcatggtgggccacgaggctctgcccatgtccttcacccagaagaccatcgaccgcctggcgggtaaacccacccacgtcaacgtgtctgtggtcatggcagaggtggacggcatctgctactaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagc

SEQ ID NO: 13 - DNA, 20 bp, Artificial Sequence
  primer
aagacctgaacaccgctccc

SEQ ID NO: 14 - DNA, 21 bp, Artificial Sequence
  primer
gttagaattgcctcagctctt

SEQ ID NO: 15 - DNA, 23 bp, Artificial Sequence
  primer
gagcccaaatcttgtgacaaaac

SEQ ID NO: 16 - DNA, 20 bp, Artificial Sequence
  primer
agcatcctcgtgcgaccgcg

SEQ ID NO: 17 - DNA, 65 bp, Artificial Sequence
  primer
ctcagtctggtccttgcactcctgtttccaagcatggcgagcatggcaatgcacgtggcccagcc

SEQ ID NO: 18 - DNA, 66 bp, Artificial Sequence
  primer
gaattcgagcccaaatcttctgacaaaactcacacatccccaccgtccccagcacctgaactcctg

SEQ ID NO: 19 - DNA, 31 bp, Artificial Sequence
  primer
tctagaagcatcctcgtgcgaccgcgagagc

SEQ ID NO: 20 - DNA, 2446 bp, Artificial Sequence
  therapeutic gene
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatgggtgtactgctcacacagaggacgctgctcagtctggtccttgcactcctgtttccaagcatggcgagcatggcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacgaattcgagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagc

SEQ ID NO: 21 - protein, 16 aa, Artificial Sequence
  N-terminal Gly is acetylated, 2nd a.a can be replaced by Ile, 4th a.a can be replaced by Leu. 10th a.a can be replaced by Arg, 11th a.a can be replaced by Lys, 13th a.a can be replaced by one of the Leu, Ile, Arg, Gln, Asn and Ser.
GLGISYGRKKRRGRRC

SEQ ID NO: 22 - DNA, 17 bp, Artificial Sequence
  Linker-1 DNA 5′ end of C forms ester bond with Cys
ctaatacgactcactat

SEQ ID NO: 23 - DNA, 16 bp, Artificial Sequence
  Linker-2 DNA
gattatgctgagtgat